(19) United States
(12) Patent Application Publication
(10) Pub. No.: US 2024/0391905 A1
(43) Pub. Date: Nov. 28, 2024

(54) BIPYRROLINONE COMPOUND

(71) Applicant: DIC Corporation, Tokyo (JP)

(72) Inventors: Yasuhiro Kuwana, Kamisu-shi (JP); Xia Yang, Qingdao (CN); Guohong Sui, Qingdao (CN); Shumei Ji, Qingdao (CN); Wei Zhao, Qingdao (CN)

(73) Assignee: DIC Corporation, Tokyo (JP)

(21) Appl. No.: 18/689,199

(22) PCT Filed: Jul. 15, 2022
(86) PCT No.: PCT/CN2022/105933

§ 371 Date: Mar. 5, 2024

(51) Int. Cl. C07D 403/14; C08K 5/3417; C09B 23/01; C09D 5/33; C09D 175/14

(52) U.S. Cl. CPC C07D 403/14; C09B 23/0075; C09D 5/004; C09D 175/14; C08K 5/3417

(57) ABSTRACT

An object is to provide bipyrrolinone compound that has a low absorption for wavelengths in the infrared region and exhibits a high degree of blackness when used as a black colorant. Another object is to provide articles including the compound, and examples of the articles include inks, printed matter, coating compositions, coated articles, plastics, fibers, films, cosmetics, and molded articles and further include articles in which any of the foregoing articles is used, such as near-infrared-transparent articles, wavelength-controlled devices, filters for infrared sensors, filters for solid-state image sensing devices, covers for LiDAR, coatings for vehicles equipped with an autonomous driving system, and black matrices for image display devices. A bipyrrolinone compound having a specific structure has been discovered, and the compound has been discovered to have a low absorption for wavelengths in the infrared region and have a high degree of blackness. Accordingly, the objects have been achieved.

### TECHNICAL FIELD

The present invention relates to a bipyrrolinone compound.

### BACKGROUND ART

In recent years, in a wide variety of fields, there has been a tendency for an increase in the number of devices, facilities, materials, and the like that require control of light having a wavelength in the infrared region. For example, such control is required for press-through package (PTP) printing for medical packaging; food packages; infrared sensors; automotive coatings; outer wall materials; road pavements; and the like.

In foreign material detection devices that are used for medical packages having printing on the PTP, an infrared sensor is used to detect foreign materials in tablets and the package itself. In this process, if a material having an absorption for wavelengths in the infrared region exists on the package, infrared light from a light source cannot be sufficiently reflected by an aluminum substrate of the package, and, consequently, the sensor recognizes that a foreign material is present. Accordingly, there is a need for the colorant used for these applications to be a material having a low absorption for wavelengths in the infrared region.

For the recycling of food packages, an infrared sensor is used, for example, to determine whether the package is recyclable and/or to identify the material of the package. If the material has an absorption for wavelengths in the infrared region, the material cannot allow infrared light from a light source to pass therethrough, or for some identification devices, the material cannot reflect the infrared light, and, consequently, the package is identified as being non-recyclable. Accordingly, there is a need for the colorant used for these applications to be a material having a low absorption for wavelengths in the infrared region.

Regarding infrared sensors, a detector, which serves as the sensor, a light source, and the like are not necessarily formed of elements that detect and/or emit only light in the infrared region. Accordingly, in instances where an infrared sensor is required to detect and/or emit only light in the infrared region, it is necessary that a material used in the sensor be a material that absorbs or reflects light in the visible region and allows only light in the infrared region to pass therethrough.

In the manufacture of a black matrix used in color filters for LCDs, OLEDs, or the like and a photo spacer for LCDs, a step after post-baking uses an infrared camera for the alignment of the photomask, and, for precise alignment, there is a need for a material that can allow light in the infrared region to pass therethrough.

Regarding automotive coatings, outer wall materials, road pavements, and the like, there is a need to prevent as much temperature increases as possible that may occur when these objects absorb sunlight during the hot season. This is an important challenge not only from the standpoint of comfort but also from the standpoint of energy conservation, and, therefore, there is a need for a material that can allow light in the near-infrared region, which is converted to heat to a large extent, to pass therethrough. Furthermore, such a material is useful for automotive coatings associated with Light Detection and Ranging (LiDAR) performed with an infrared laser system; LiDAR is used for surrounding environment distance measurement for a vehicle equipped with an autonomous driving system, which is expected to be widely used in the future.

In the above-described exemplary applications, there is a trend toward using not only chromatic colors but also black for the exterior, and there are various reasons for the trend. For printing on PTPs and food packages, black is used primarily for character recognition; for infrared sensors, black is used primarily to conceal the sensors themselves; for automotive coatings and outer wall materials, black is a popular color and is, therefore, used primarily to satisfy personal preferences; and for road pavements, black is a color of asphalt. Thus, although the reasons for the use differ, black, which is an achromatic color, is a basic color and used in many applications like white, without being limited to the above-mentioned applications.

As described, it is desirable that black colorants for the above-mentioned applications basically be materials that can allow light having a wavelength in the infrared region to pass therethrough. However, carbon black, which is a commonly used black colorant, absorbs light having wavelengths in a wide range of the infrared region, including light having wavelengths of 800 to 1400 nm, which is converted to heat to a large extent, and, therefore, there is a need for a black colorant that can replace carbon black.

One known black colorant is a bipyrrolinone-based pigment (PTL 1), which has been proven to provide a black coloration. However, the bipyrrolinone-based pigment has a significant degree of absorption for wavelengths in the infrared region and, therefore, presents a problem associated with infrared transparency. Furthermore, Pigment Black 31 and Pigment Black 32, which are known as perylene black pigments, provide a black coloration when used at a high concentration, but the pigments provide a dark green coloration when used at a low concentration. Accordingly, these pigments can be used only in a limited manner.

### CITATION LIST

### Patent Literature

[PTL 1] Japanese Unexamined Patent Application Publication No. 2009-84522

### SUMMARY OF INVENTION

### Technical Problem

An object of the present invention is to provide a bipyrrolinone compound that has a low absorption for wavelengths in the infrared region and exhibits a high degree of blackness when used as a black colorant. Another object of the present invention is to provide articles including the compound, and examples of the articles include inks, printed matter, coating compositions, coated articles, plastics, fibers, films, cosmetics, and molded articles and further include articles in which any of the foregoing articles is used, such as near-infrared-transparent articles, wavelength-controlled devices, filters for infrared sensors, filters for solid-state image sensing devices, covers for LiDAR, coatings for vehicles equipped with an autonomous driving system, and black matrices for image display devices.

### Solution to Problem

The inventors diligently performed studies to achieve the object and, consequently, discovered a bipyrrolinone compound having a specific structure. The inventors discovered that the compound has a low absorption for wavelengths in the infrared region and has a high degree of blackness and, accordingly, achieved the object.

Specifically, the present invention encompasses the items described below.

(1) A compound represented by general formula (1) below.

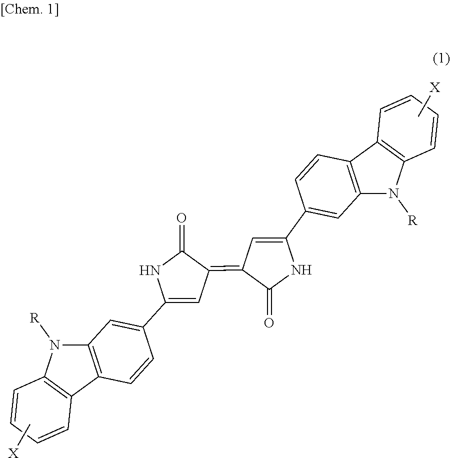

In the formula, R represents an alkyl group having 1 to 3 carbon atoms or represents an alkenyl group having 2 to 3 carbon atoms, and X represents one of a nitro group, a cyano group, a halogen, and an acetyl group.

(2) The compound according to (1), wherein the compound is a bipyrrolinone compound.

(3) The compound according to (1) or (2), wherein the compound is transparent to light having a wavelength of 800 to 1400 nm.

(4) An ink, printed matter, a coating composition, a coated article, a plastic, a fiber, a film, a cosmetic, and a molded article that include the compound according to any one of (1) to (3) and are transparent to light in a near-infrared region.

(5) An ink, printed matter, a coating composition, a coated article, a plastic, a fiber, a film, a cosmetic, and a molded article that include the compound according to any one of (1) to (3), the compound serving as a colorant, a coloring agent, or a pigment.

(6) An ink, printed matter, a coating composition, a coated article, a plastic, a fiber, a film, a cosmetic, and a molded article that include the compound according to any one of (1) to (3), the compound serving as a colorant, a coloring agent, or a pigment that is transparent to light in a near-infrared region.

### Advantageous Effects of Invention

The bipyrrolinone compound of the present invention has a low absorption for wavelengths in the infrared region and has a high degree of blackness. Since the compound has a low absorption for wavelengths in the infrared region and has a high degree of blackness for various applications, the compound can be used in a wide variety of industrial fields. Specifically, the applications include wavelength-controlled devices including the compound, such as devices that require a near-infrared transparency, examples of which include filters for infrared sensors, filters for solid-state image sensing devices, covers for LiDAR, and coatings for vehicles equipped with an autonomous driving system, and the applications further include articles that are used in the foregoing devices, such as inks, printed matter, coating compositions, coated articles, plastics, fibers, films, cosmetics, and molded articles.

### BRIEF DESCRIPTION OF DRAWINGS

FIG. 1 is a reflection spectrum of a bipyrrolinone compound of the present invention.

### DESCRIPTION OF EMBODIMENTS

The below-described embodiments of the present invention are merely some of the embodiments of the present invention. Embodiments are not limited to the descriptions below provided that the embodiments do not significantly depart from the scope of the present invention.

### [Bipyrrolinone Compound]

A bipyrrolinone compound of the present invention is a compound represented by general formula (1) below.

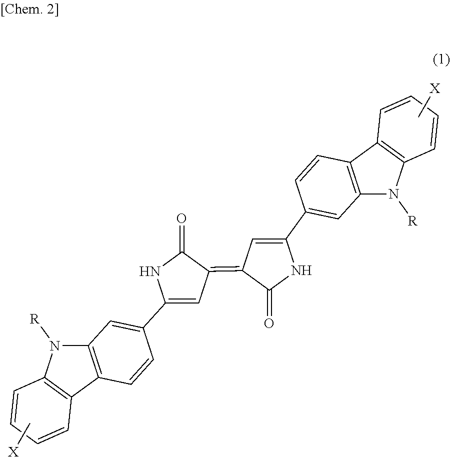

In the formula, R represents an alkyl group having 1 to 3 carbon atoms or represents an alkenyl group having 2 to 3 carbon atoms, and X represents one of a nitro group, a cyano group, a halogen, and an acetyl group.

A bipyrrolinone compound is a compound having a 5-membered ring unsaturated lactam skeleton represented by general formula (1-1) below.

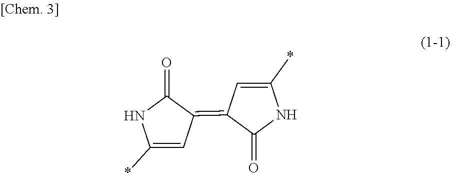

Examples of bipyrrolinone compounds include compounds in which an aromatic compound, such as a phenyl group or a biphenyl group, is attached at the positions indicated by the asterisks.

The bipyrrolinone compound of the present invention contains a substituted carbazole compound attached at the positions indicated by the asterisks in general formula (1-1). In general formula (1), R is preferably an ethenyl group, a propenyl group, an ethenyl group, or a propenyl group and particularly preferably an ethyl group or an ethenyl group. X is preferably a nitro group, a cyano group, chlorine, or bromine and particularly preferably a nitro group or a cyano group.

Examples of the bipyrrolinone compound of the present invention include, but are not limited to, the compounds shown below.

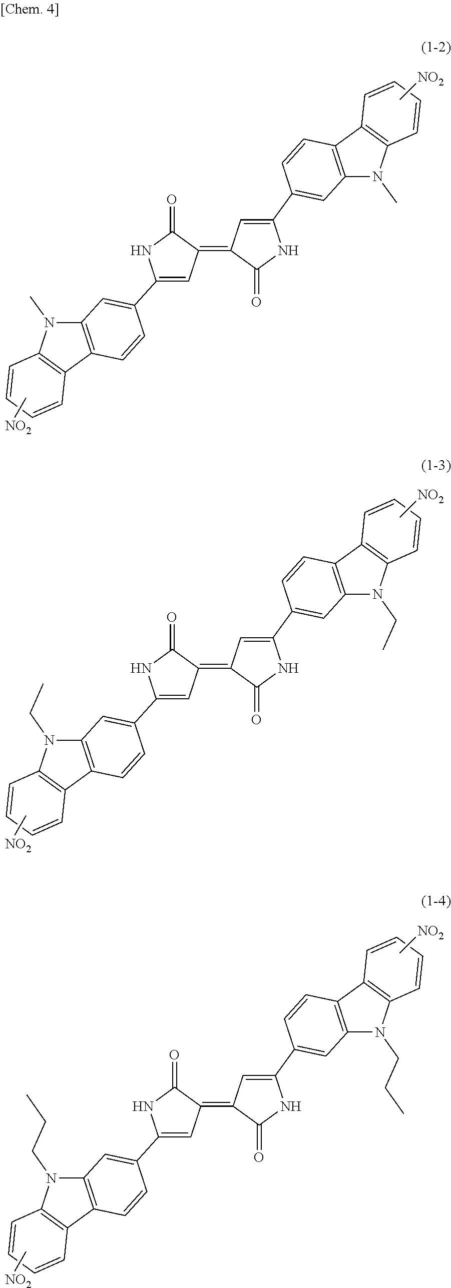

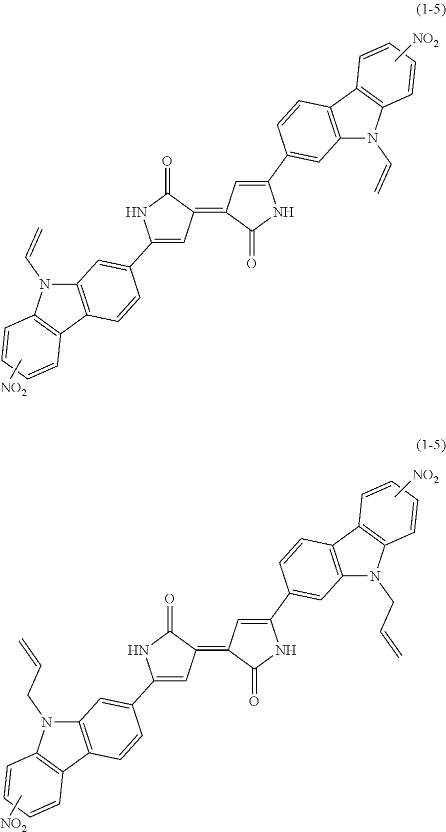

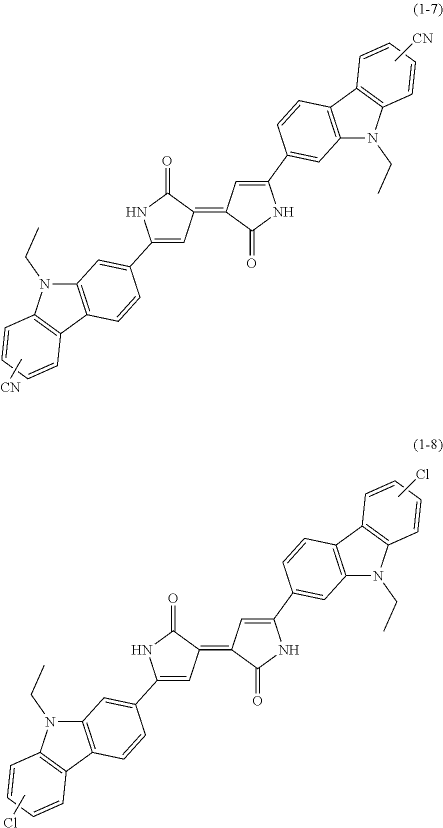

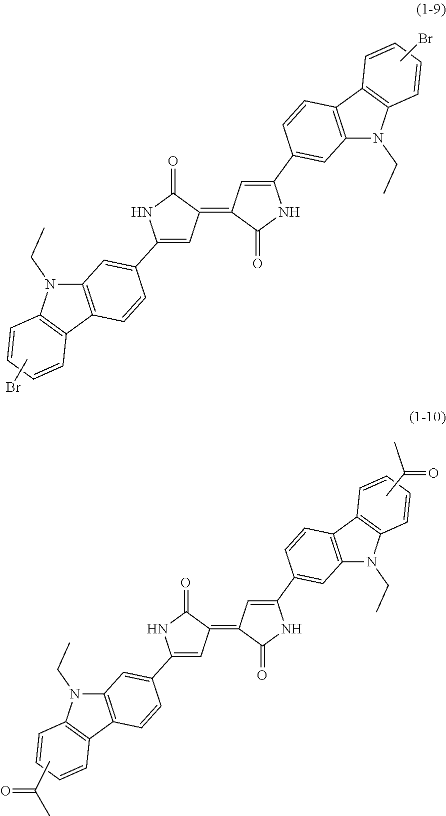

One of these compounds may be used alone, or a mixture of two or more of these compounds may be used.

### [Method for Producing Bipyrrolinone Compound]

The bipyrrolinone compound of the present invention is produced by a production method known in the art. For example, a method for producing the bipyrrolinone compound may be a method that uses a β-aryloyl propionic acid compound as a starting material. The β-aryloyl propionic acid compound can be obtained by using any of various commonly used methods. An example of a simple method is acylation of an arene with succinic anhydride. The acylation reaction is carried out in a solvent, such as nitrobenzene, dichloroethane, or carbon disulfide, in the presence of a Lewis acid catalyst, such as aluminum chloride, iron chloride, iron bromide, or tin chloride.

The production of a bipyrrolinone-based compound can be carried out as follows. A pyrrolinone compound is prepared by converting the β-aryloyl propionic acid compound to an enamine and subsequently cyclodehydrating the enamine in a homogeneous system containing an oxidizing agent, such as nitrobenzene, or in a dehydrating agent containing acetic acid, such as acetic anhydride, and then, the pyrrolinone compound is dimerized by oxidation. With this method, side reactions can be inhibited, and also, for example, an additional step of changing reaction vessels in the process of production of the final product from the raw material is not necessary, which results in a reduced yield loss.

Specifically, the β-aryloyl propionic acid compound is converted to an enamine and subsequently cyclodehydrated in a homogeneous system containing an oxidizing agent.

With this method, in instances where the reaction is controlled at a relatively low temperature, only a pyrrolinone-based compound can be selectively produced, whereas, as will be described later, in instances where the reaction is controlled at a relatively high temperature, only a bipyrrolinone-based compound can be selectively produced, via the production of a pyrrolinone-based compound.

The oxidizing agent used in the method is a liquid or solid oxidizing agent that can form a homogeneous system when the reaction raw materials are loaded. The oxidizing agent is an agent that has a function of oxidizing the pyrrolinone-based compound, which will be described later. Examples of the oxidizing agent include peroxides, such as hydrogen peroxide and m-chloroperoxybenzoic acid (mCPBA); nitro compounds, such as nitrobenzene; quinone compounds, such as chloranil and 2,3-dichloro-5,6-dicyano-1,4-benzoquinone (DDQ); sulfoxide compounds, such as dimethyl sulfoxide (DMSO); metal oxides, such as chromic acid, manganese dioxide, and selenium dioxide; and metal salts, such as lead tetraacetate.

The oxidizing agent can be used in a ratio of 1 or greater relative to the β-aryloyl propionic acid compound on an equivalent weight basis. To form a homogeneous system, an organic solvent that dissolves the oxidizing agent may be used in combination with the oxidizing agent, or in instances where the oxidizing agent is one that is liquid at room temperature, the oxidizing agent may be used in an amount greater than the amount necessary for the oxidizing agent to perform an oxidizing function; in this case, the reaction system can be a system in which the oxidizing agent also serves as a solvent. In instances where the oxidizing agent is used in this manner, it is preferable that the oxidizing agent be used in an amount of 50 to 2000 parts per 100 parts of the β-aryloyl propionic acid compound, on a mass basis.

It is preferable that the oxidizing agent be an organic oxidizing agent. This is because, in this case, a solubility of the oxidizing agent in an organic solvent can be easily increased. Furthermore, in terms of safety, it is preferable to avoid using a large amount of an oxidizing agent having a high oxidation property. From these standpoints, methods that tend to form an inhomogeneous system, such as a method that uses air or oxygen as the oxidizing agent and injects the air or oxygen into the reaction system, may not be preferable, and preferred methods are methods using a nitro compound or a quinone compound because these compounds have mild reactivity and a high safety property even when used in an excessive amount. A nitro compound, in particular, nitrobenzene, is preferable in terms of availability of the raw material.

In the step of producing the pyrrolinone-based compound, the β-aryloyl propionic acid compound is converted to an enamine and subsequently cyclodehydrated in the reaction system described above.

The enamination of the β-aryloyl propionic acid compound is carried out with an aminating agent. The aminating agent may be ammonia, an amine, or a compound that produces ammonia or an amine during the reaction. Examples of such a compound include ammonia gases, liquid ammonia, ammonium salts, such as ammonium acetate and ammonium hydrochloride, urea, and primary amines, such as methylamine, ethylamine, and n-butyl amine. In instances where the aminating agent is a solid or a liquid, the aminating agent may be directly loaded into the reaction system, or in instances where the aminating agent is a gas, the aminating agent may be bubbled into the reaction system.

To increase the degree of conversion of the enamination reaction, it is preferable that the aminating agent be used in an excessive amount relative to the amount of the β-aryloyl propionic acid compound. In instances where the aminating agent is ammonium acetate, the ammonium acetate may be used in an amount of 2 to 2.5 moles per mole of the β-aryloyl propionic acid compound.

The enamination of the β-aryloyl propionic acid can be carried out by mixing and stirring the β-aryloyl propionic acid and an aminating agent together in an organic solvent, which is optionally used. The organic solvent that may be used may be an oxidizing agent that is liquid at room temperature or a dehydrating agent that is liquid at room temperature, mentioned above.

The enamination reaction may be carried out at a temperature of 50 to 200° C. for a period of 1 to 50 hours, for example.

Furthermore, a catalyst, such as acid, may be used to improve the reaction rate for the enamination. Examples of acidic catalysts include organic acids, such as para-toluenesulfonic acid and acetic acid, and inorganic acids, such as hydrochloric acid, sulfuric acid, and nitric acid. Since an acidic catalyst forms a salt with the aminating agent, in instances where a strongly acidic catalyst is used, the catalyst may be used in a catalytic amount, for example, 0.05 to 0.5 moles per mole of the β-aryloyl propionic acid.

Furthermore, in instances where the acidic catalyst is a weakly acidic catalyst, such as acetic acid, the salt formed with the aminating agent can reversibly produce an amine during the reaction, and, accordingly, the catalyst may be used in an amount of 1 to 1.5 moles per mole of the β-aryloyl propionic acid.

The progress of the enamination causes water to be liberated. Since the water hydrolyzes the produced enamine, it is preferable that the water be removed from the reaction system, for example, with a Dean-Stark trap or the like.

The enamine intermediate produced by the amination dehydrates and condenses with the carboxyl group within the molecule to give a pyrrolinone compound. This intramolecular dehydration condensation reaction can be accelerated, for example, with an acidic catalyst. Accordingly, it is preferable that an acidic catalyst, such as one of those mentioned above, be loaded into the reaction system in advance. Examples of the acidic catalyst include the above-mentioned acidic catalysts used for the enamination.

Furthermore, in the intramolecular dehydration condensation reaction, too, it is preferable that the water produced by the intramolecular dehydration be removed from the reaction system, as with the enamination reaction, so that the equilibrium can be shifted to the production side.

For the cyclodehydration reaction, a heating temperature may be 60 to 170° C., and a heating time may be 30 minutes to 10 hours, for example.

Reaction temperatures for the enamination and the cyclodehydration may preferably be 80 to 150° C. in instances in which the final target product is a pyrrolinone-based compound.

In the method for producing a pyrrolinone-based compound, the endpoint of the reaction can be ascertained, for example, by subjecting the reaction liquid to one or more chromatography steps and determining the point at which an amount of the pyrrolinone-based compound produced no longer increases.

The pyrrolinone-based compound produced in this manner may be filtered and dried to be used in any suitable form. Furthermore, the pyrrolinone-based compound may be purified, for example, by subjecting the pyrrolinone-based compound to washing and recrystallization.

Now, a method for producing the bipyrrolinone-based compound will be described.

The bipyrrolinone-based compound can be produced by oxidizing the pyrrolinone-based compound produced by the method described above. This reaction is a reaction that produces one mole of a bipyrrolinone-based compound from two moles of a pyrrolinone-based compound.

In instances where the pyrrolinone-based compound is produced by the method described above, a substance that serves as an oxidizing agent is already present in the reaction system after completion of the reaction, and, therefore, in the production of the bipyrrolinone-based compound, oxidation can be accomplished without the need to add an oxidizing agent to the reaction system.

Presumably, a molecule of the pyrrolinone-based compound becomes 2,3-diketopyrroline as a result of oxidation of the methylene carbon at the alpha position of the carbonyl group, and the 2,3-diketopyrroline undergoes intermolecular dehydration condensation with another molecule of the pyrrolinone-based compound to form the bipyrrolinone-based compound. In the production of the bipyrrolinone-based compound from the pyrrolinone-based compound, it is preferable to avoid using an excessive amount of an oxidizing agent having a high oxidation property, so that a decrease in the yield of the bipyrrolinone-based compound can be prevented; the decrease may otherwise occur if the amount of the pyrrolinone-based compound that reacts with 2,3-diketopyrroline, which is produced by the oxidation of the pyrrolinone-based compound, becomes insufficient. Accordingly, it is preferable to employ a method that uses an oxidizing agent having a mild reactivity, being capable of more homogeneous oxidation, and having a high safety property, as with the method described above.

The oxidation of the pyrrolinone-based compound may be carried out in the presence of an organic solvent. Examples of the organic solvent include aromatic hydrocarbon-based solvents, and examples of the aromatic hydrocarbon include toluene and xylene.

For the oxidation reaction of the pyrrolinone-based compound into the bipyrrolinone-based compound, a heating temperature may be 70 to 250° C., and a heating time may be 1 to 50 hours, for example. Step-wise heating may be employed, in which a constant temperature is maintained for a certain period of time, and thereafter, the temperature is increased.

In the oxidation reaction, it is preferable to stir the reaction mixture to prevent bumping of the water produced by the dehydration condensation reaction. Regarding the reaction temperature, in instances where nitrobenzene is used as an oxidizing agent and reaction solvent, heating at a temperature of 50 to 250° C. is preferable in terms of improving the oxidation reaction rate, and heating at a temperature of 100 to 220° C. is preferable in terms of accelerating the dehydration condensation reaction. Furthermore, heating at a temperature of 155 to 200° C. can further improve the rate at which the bipyrrolinone-based compound is produced.

In the method for producing the bipyrrolinone-based compound, a process may be employed in which a homogeneous system containing an oxidizing agent in an amount sufficient for the oxidizing agent to serve as an oxidizing agent and reaction solvent is used, and the reaction is carried out at a relatively low temperature (e.g., 50 to 150° C.); thus, by oxidizing the resulting pyrrolinone-based compound, a bipyrrolinone-based compound can be immediately obtained.

The endpoint of the oxidation reaction of the pyrrolinone-based compound can be ascertained, for example, by subjecting the reaction liquid to one or more chromatography steps and determining the point at which an amount of the bipyrrolinone-based compound produced no longer increases.

The bipyrrolinone-based compound produced in this manner may be filtered and dried to be used in any suitable form. Furthermore, the bipyrrolinone-based compound may be purified, for example, by subjecting the bipyrrolinone-based compound to washing and recrystallization. In addition, the bipyrrolinone-based compound may be subjected to a refining process and/or one or more surface treatments, which enable the bipyrrolinone-based compound to be used as a colorant, a coloring agent, or a pigment suitable for coloring an object that is to be colored.

After the production, the bipyrrolinone compound produced by the production method has a large particle size and a non-uniformity of the particles in many cases, and, therefore, the bipyrrolinone compound exhibits poor dispersibility for some applications. Accordingly, depending on the need, an additional step of controlling the particles is necessary so that a desired particle size and crystal form can be achieved.

In instances where control of the particles is to be performed, any of a variety of commonly known methods may be used. Specific examples of the methods include a method in which the bipyrrolinone compound of the present invention is kneaded and milled with a water-soluble inorganic salt and a water-soluble organic solvent (solvent salt milling method); a method in which the bipyrrolinone compound of the present invention is heated in a solvent in which the bipyrrolinone compound is insoluble (solvent method); and a method in which the bipyrrolinone compound is finely ground in a grinder or disperser.

An example of the solvent salt milling method is a method in which the bipyrrolinone compound of the present invention is kneaded and milled with a water-soluble inorganic salt and a water-soluble organic solvent under heat, and the resulting particles are washed with water; examples of the water-soluble inorganic salt include sodium chloride and sodium sulfate, and examples of the water-soluble organic solvent include diethylene glycol and triethylene glycol.

In instances where the solvent method is performed, the liquid medium to be used is one that does not dissolve the bipyrrolinone compound of the present invention. The liquid medium may be a liquid medium including, as an essential component, a water-soluble organic solvent. This is preferable in terms of controlling the crystallinity of the bipyrrolinone compound of the present invention more consistently.

In instances where a method for fine grinding is performed, a pigment mill or a pigment disperser may be used, examples of which include ball mills, sand mills, attritors, horizontal continuous medium dispersers, kneaders, continuous single screw kneaders, continuous twin screw kneaders, three-roll mills, and open roll continuous kneaders. These mills and dispersers can also be used in the solvent salt milling method.

In instances where the particles are controlled, the bipyrrolinone compound of the present invention can exhibit crystalline properties rather than molecular properties. Specifically, when the bipyrrolinone compound has crystalline properties, the bipyrrolinone compound can maintain blackness, robustness, and wavelength controllability at high levels.

### [Near-Infrared-Transparent Articles]

The bipyrrolinone compound of the present invention can be used in articles that are required to be near-infrared-transparent, and examples of the articles include inks, printed matter, coating compositions, coated articles, plastics, fibers, films, cosmetics, and molded articles.

For example, the bipyrrolinone compound is suitable for use in filters for infrared sensors, filters for solid-state image sensing devices, covers for LiDAR, coatings for vehicles equipped with an autonomous driving system, black matrices for image display devices, and the like.

The bipyrrolinone compound of the present invention can be used in articles that use an infrared sensor, and reasons for this are as follows. In devices equipped with an infrared sensor, infrared transparency is necessary, depending on the application and the type of the device. Specifically, on the oscillator side from which infrared light is emitted, infrared transparency is necessary for emitting the generated infrared light to the outside of the device, and on the detector side at which the infrared light is received, infrared transparency is necessary for preventing the infrared light from the outside from being blocked. Furthermore, for these devices, there is a trend toward a preference for the black color, and a reason for this is that, for example, the black color has functions of protecting the oscillator and the light receiver, concealing the fact that the article itself is an infrared sensor, and obscuring the presence of the article itself.

The bipyrrolinone compound of the present invention can be used in articles for solid-state image sensing device applications, and reasons for this are as follows. Functions similar to those for infrared sensors are desired. Furthermore, in addition to the functions associated with infrared sensors, increased light resistance is desired because solid-state image sensing devices are more frequently used outdoors, and, accordingly, for these devices, there is a trend toward a preference for pigment-based coloring matter to dye-based coloring matter.

The bipyrrolinone compound of the present invention can be used in articles for LiDAR applications, and examples of the use are as follows. In one example, the bipyrrolinone compound is used for a light detection and ranging (LiDAR) function, which corresponds to that of infrared sensors, and in another example, the bipyrrolinone compound is used in a coating film of a target object of the LiDAR distance measurement. Particular examples of the target object include vehicles such as automobiles and trucks.

A main principle of LiDAR is the time of flight (TOF) principle, which is to calculate a distance and a direction to a target object and a shape of the target object through a process in which emitted laser light reaches the target object, and the laser light reflected off the object is received by a photosensor.

Articles including the bipyrrolinone compound of the present invention can be used in LiDAR devices themselves, and reasons for this are similar to those discussed above for infrared sensors. Furthermore, an additional reason is as follows. For LiDAR devices, since the wavelength of the infrared sensor used as the light source is, for example, 905 nm or 1550 nm, a transparency to only these wavelengths may be sufficient; however, since bandpass filters are excessive, there is a trend toward a preference for a material transparent to wavelengths in the entire near-infrared region.

Articles including the bipyrrolinone compound of the present invention can be used in coating films of vehicles. In this instance, one premise is that infrared light needs to be reflected by the chassis or the body itself of the vehicle or by a primer layer or an underlayer of the coating. When laser light reaches a vehicle, the laser light passes through the infrared-transparent coating film and is reflected by a primer layer or an underlayer, and subsequently, the laser light passes through the coating film again to be emitted out of the vehicle. In general, for vehicles, there is a trend toward a preference for a coloration provided by the use of a basic color such as a white-based color or a black-based color.

The bipyrrolinone compound of the present invention can be used in black matrices for image display devices and in other similar articles, and reasons for this are as follows. High opaqueness for the visible light region is required because color mixing of the RGB colors, which may occur when visible light from a light source of any of various types passes through a color filter, needs to be prevented. Furthermore, for black matrix applications, there is a trend toward a preference for dyes and pigments having the following characteristics, which are associated with the manufacturing process for black matrices. The manufacturing process includes a photolithography process, which is performed after the application of materials to a substrate and drying of the materials, and the photolithography process includes, for example, exposure that uses a photomask, development, and post-baking. The characteristics are a degree of transparency to the UV region with which a degree of photocurability can be obtained for the exposed portions in exposure that uses a photomask and heat resistance sufficient to retain opaqueness for the process of post-baking.

### [Colorant Applications]

With the use of the bipyrrolinone compound of the present invention, it is possible to provide articles such as inks, printed matter, coating compositions, coated articles, plastics, fibers, films, cosmetics, and molded articles, and moreover, in instances where these articles are required to be near-infrared-transparent, the need can be satisfied. The uses discussed in detail below are merely examples. Application of the bipyrrolinone compound of the present invention enables a variety of uses.

### [Ink Applications]

The bipyrrolinone compound of the present invention can be used in printing inks. The printing inks can be prepared by mixing one or more commonly known binder resins, solvents, additives, and the like with the bipyrrolinone compound of the present invention in accordance with a preparation method of the related art. Specifically, a liquid ink can be prepared by preparing a high-pigment-concentration base ink for a liquid ink and adding one or more binders, solvents, additives, and/or the like to the base ink.

With the use of the bipyrrolinone compound of the present invention, PU inks and NC inks can be produced. Accordingly, the bipyrrolinone compound is suitable for use in an organic pigment composition for gravure printing inks and flexographic printing inks. PU inks include a PU resin, a pigment, a solvent, and one or more additives. NC inks include an NC resin, a pigment, a solvent, and one or more additives. The PU resin is not particularly limited as long as the PU resin has a urethane structure in the backbone. Examples of the PU resin include polyurethanes and polyureas. Examples of the solvents include aromatic organic solvents, such as toluene and xylene; ketone-based solvents, such as methyl ethyl ketone, methyl isobutyl ketone, cyclohexanone, 2-heptanone, and 3-heptanone; ester-based solvents, such as ethyl acetate, n-propyl acetate, isopropyl acetate, isobutyl acetate, propylene glycol monoethyl ether acetate, and propylene glycol monomethyl ether acetate; alcoholic solvents, such as methanol, ethanol, n-propanol, isopropanol, n-butanol, isobutanol, and t-butanol; (poly)alkylene glycol monoalkyl ether-based solvents, such as propylene glycol monoethyl ether, propylene glycol monomethyl ether, ethylene glycol monomethyl ether, ethylene glycol monoethyl ether, ethylene glycol mono-n-propyl ether, ethylene glycol mono-i-propyl ether, diethylene glycol monomethyl ether, diethylene glycol monoethyl ether, diethylene glycol mono-n-propyl ether, and diethylene glycol mono-i-propyl ether; (poly)alkylene glycol monoalkyl ether acetate-based solvents, such as ethylene glycol monomethyl ether acetate, ethylene glycol monoethyl ether acetate, diethylene glycol monomethyl ether acetate, and diethylene glycol monoethyl ether acetate; and other ether-based solvents, such as diethylene glycol dimethyl ether and diethylene glycol diethyl ether. Note that one solvent may be used alone, or two or more solvents may be used in combination. Examples of the one or more additives include surfactants, such as anionic surfactants, nonionic surfactants, cationic surfactants, and zwitterionic surfactants; rosins, such as gum rosins, polymerized rosins, disproportionated rosins, hydrogenated rosins, maleated rosins, hardened rosins, and phthalic alkyd resins; pigment derivatives; dispersants; humectants; adhesion aids; leveling agents; defoaming agents; antistatic agents; trapping agents; anti-blocking agents; and wax components.

In the instance where the bipyrrolinone compound of the present invention is used in a printing ink, the printing ink including the bipyrrolinone compound of the present invention, which is prepared as described above, may be used by being diluted in ethyl acetate, polyurethane-based varnish, or polyamide-based varnish. The preparation of the printing ink may be carried out by using a commonly known method.

### [Coating Composition Applications]

In instances where the bipyrrolinone compound of the present invention is used in a coating composition, a resin to be used in the coating composition may be any of a variety of resins, examples of which include acrylic resins, melamine resins, epoxy resins, polyester resins, polyurethane resins, polyamide resins, and phenolic resins.

Examples of solvents that may be used in the coating composition include aromatic solvents, such as toluene, xylene, and methoxy benzene; acetate-based solvents, such as ethyl acetate, butyl acetate, propylene glycol monomethyl ether acetate, propylene glycol monoethyl ether acetate; propionate-based solvents, such as ethoxy ethyl propionate; alcoholic solvents, such as methanol, ethanol, propanol, n-butanol, and isobutanol; ether-based solvents, such as butyl cellosolve, propylene glycol monomethyl ether, diethylene glycol ethyl ether, and diethylene glycol dimethyl ether; ketone-based solvents, such as methyl ethyl ketone, methyl isobutyl ketone, and cyclohexanone; aliphatic hydrocarbon-based solvents, such as hexane; nitrogen compound-based solvents, such as N,N-dimethylformamide, γ-butyrolactam, N-methyl-2-pyrrolidone, aniline, and pyridine; lactone-based solvents, such as γ-butyrolactone; carbamic acid esters, such as a mixture of methyl carbamate and ethyl carbamate mixed at a ratio of 48:52; and water. Particularly suitable solvents are polar solvents that are soluble in water, such as propionate-based solvents, alcoholic solvents, ether-based solvents, ketone-based solvents, nitrogen compound-based solvents, lactone-based solvents, and water.

Furthermore, in instances where a resin composition for a coating composition is prepared by dispersing or mixing a pigment additive and/or a pigment composition in a liquid resin, one or more typical additives may be added to the liquid resin. Examples of the typical additives include dispersants, fillers, coating auxiliary agents, drying agents, plasticizing agents, and auxiliary pigments. The components may be added singly or in combinations of several components to incorporate all the components, or all the components may be added at one time. The components are then dispersed or mixed together to form the resin composition for a coating composition.

Regarding a disperser for dispersing the composition containing the bipyrrolinone compound, which is prepared as described above in accordance with the use, the disperser may be a known disperser, examples of which include, but are not limited to, dispers, homogenizers, paint conditioners, Scandex dispersers, bead mills, attritors, ball mills, two-roll mills, three-roll mills, and pressure kneaders. A resin and a solvent are added to the pigment composition in a manner such that the pigment composition has a viscosity that enables the pigment composition to be dispersed with any of these dispersers. After the dispersing operation, the high-concentration base for a coating composition has a solids content of 5 to 20%. A resin and a solvent are further mixed with the base to provide a coating composition for use.

### [Plastics Applications]

The bipyrrolinone compound of the present invention can be used in plastic coloring applications. In instances where a colored plastic molded article is produced, a thermoplastic resin (plastic) for heat molding, which may be injection molding, press molding, or the like, is used, and examples of the thermoplastic resin include polyolefins, such as polyethylene and polypropylene, and polyvinyl chloride resins. A pigment of the present invention can be used by being kneaded into the resin by using a method known in the art.

### [Cosmetics Applications]

The bipyrrolinone compound of the present invention can be used in cosmetics. The cosmetics in which the bipyrrolinone compound can be used are not particularly limited. The bipyrrolinone compound of the present invention can be used in various types of cosmetics.

The cosmetics may be any of various types of cosmetics as long as the functions of the cosmetics can be exhibited effectively. The cosmetics may be in the form of a lotion, a cream gel, a spray, or the like. Examples of the cosmetics include skin-care cosmetics, such as facial cleansers, makeup removers, skin lotions, serums, packs, protective milky lotions, protective creams, skin whiteners, and UV-protective cosmetics; makeup, such as foundations, face powders, makeup bases, lipsticks, eye makeup, blushers, and nail enamels; hair-care cosmetics, such as shampoos, hair rinses, hair treatments, hair styling products, permanent wave agents, hair dyes, and hair tonics; and body-care cosmetics, such as body cleansing cosmetics, deodorant cosmetics, and bath salts.

A content of the bipyrrolinone compound of the present invention used in these cosmetics may be appropriately set in accordance with the type of the cosmetic. The content in any of the above-mentioned cosmetics is typically within a range of 0.1 to 99 mass %. In general, the content is preferably within a range of 0.1 to 10 mass %. On the other hand, in makeup used for coloring, the content is preferably within a range of 5 to 80 mass %, more preferably 10 to 70 mass %, and most preferably 20 to 60 mass %. When the content of the bipyrrolinone compound of the present invention present in the cosmetic is within any of the above-mentioned ranges, functions thereof, such as a coloring property, can be exhibited effectively, and functions required of a cosmetic can be maintained.

The cosmetics may include, in addition to the bipyrrolinone compound of the present invention, one or more components that are permissible as cosmetic components, depending on the type of cosmetic. Examples of the one or more components include supports, pigments, oils, sterols, amino acids, powders, coloring agents, pH adjusting agents, perfumes, essential oils, cosmetic active ingredients, vitamins, essential fatty acids, sphingolipids, self-tanning agents, excipients, fillers, emulsifiers, antioxidants, surfactants, chelating agents, gelling agents, thickening agents, emollient agents, humectants, moisturizing agents, minerals, viscosity modifiers, rheology modifiers, keratolytic agents, retinoids, hormonal compounds, alpha-hydroxy acids, alpha-keto acids, anti-mycobacterial agents, antifungal agents, antimicrobial agents, antiviral agents, painkillers, antiallergic agents, antihistamines, anti-inflammatory agents, anti-irritant agents, antitumor agents, immune system boosting agents, immune system suppressing agents, anti-acne agents, anesthetic agents, disinfectants, insect repellents, skin cooling compounds, skin protectants, skin penetration enhancers, exfoliants, lubricants, fragrances, staining agents, bleaching agents, hypopigmenting agents, antiseptic agents, stabilizers, pharmaceutical products, light stabilizers, and spherical powders.

The cosmetics can be produced by mixing the bipyrrolinone compound of the present invention with one or more other cosmetic components.

Furthermore, cosmetics including the bipyrrolinone compound of the present invention can be used in a manner similar to that of typical cosmetics in accordance with the type of cosmetic, for example.

### EXAMPLES

The present invention will now be described in more detail with reference to examples. Note that the present invention is not limited to the examples. Furthermore, “%” used for the compositions of the examples below is “mass %”.

### Synthesis Example 1

7.4 g of 3-nitroethyl carbazole and 3.0 g of succinic anhydride were mixed with 85 g of nitrobenzene under nitrogen purging, and 8.5 g of aluminum chloride was further added. The resulting mixture was stirred at room temperature for 2 hours, and subsequently, the contents were allowed to react at 60° C. for 4 hours. After the mixture was returned to room temperature, 17 ml of concentrated hydrochloric acid and 170 g of water were added to the reaction solution, which was then stirred at room temperature for 2 hours. Subsequently, the resultant was filtered, washed with water, and dried to give 5.67 g of an intermediate 1. The obtained intermediate 1 had a purity of 94.5%.

Next, 4.08 g of the obtained intermediate 1 was mixed with a solution containing 1.85 g of ammonium acetate and 50 g of nitrobenzene, and the mixture was stirred at 90° C. for 2 hours and further stirred at 125° C. for 1 hour. The resulting mixture was heated at 200° C. for 4 hours to remove water therefrom, and subsequently, the resultant was cooled to give a blackish green precipitate. The resulting precipitate was washed and filtered repeatedly, with the washing being performed with 100 ml of acetone twice and further performed with 50 ml of methanol and water. The resulting wet cake was added to DMF, which was then refluxed for 2 hours. Subsequently, the resultant was washed with DMF, acetone, and water and then dried to give 2.17 g of a black compound. The yield was 57%. Furthermore, electron impact mass spectrometry (MS-EI) was conducted, and the results confirmed that the compound had a molecular weight of 638 (m/z=638).

The obtained black compound was salt-milled with 10.85 g of sodium chloride and 2.5 g of diethylene glycol, the resultant was washed with water and filtered repeatedly, and the resulting wet cake was dried to give 2.01 g of a black compound of Example 1.

Note that the instrument and conditions used for the MS-EI spectrometry are as follows.
Name of instrument: 5973N GC/MS, manufactured by Agilent Technologies Inc.

### Measurement Conditions Conditions for GC

Initial temperature: 50° C.
Heating Rate: 100° C./minute (target temperature: 450° C.)

### Conditions for MS

Mass range: 50 to 800
Quadrupole temperature: 150° C.
Inert ion source temperature: 230° C.

An infrared absorption of the obtained black compound was measured with a Fourier-transform infrared spectrophotometer (FT-IR), and the results were that an absorption peak corresponding to the NH linkage was observed at 3395 cm−1, an absorption peak corresponding to the carbonyl linkage at 1675 cm−1, and an absorption peak corresponding to the nitro group at 1321 cm−1.

Note that the instrument and conditions used for the FT-IR spectrometry are as follows.
Name of instrument: FT/IR-6100, manufactured by JASCO Corporation
Measurement Conditions Form of sample: powder
Measurement mode: attenuated total reflection (ATR)
Measurement wavelengths: 400 to 4000 cm−1

A crystallinity of the obtained black compound was measured with an X-ray diffractometer, and the results were that X-ray diffraction peaks were observed at 8.8°, 11.0°, 14.5°, 17.6°, 21.8°, 23.9°, 25.3°, 27.1°, and 29.2°. Note that the instrument and conditions used for the X-ray diffraction measurement are as follows.
Name of instrument: X-ray diffractometer MiniFlex II, manufactured by Rigaku Corporation
Measurement Conditions Form of sample: powder
Measurement angle: 2 to 40°, scan speed: 2°/minute

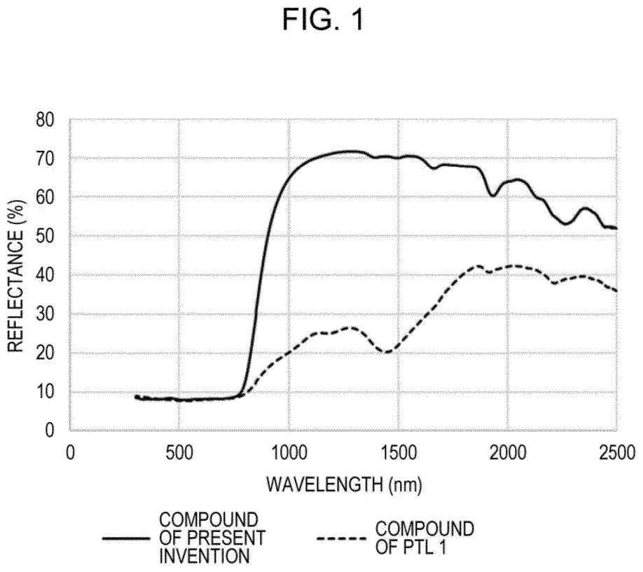

Next, a near-infrared transparency of the obtained black compound was measured with a spectrophotometer. The results are shown in FIG. 1, which indicates that the bipyrrolinone compound of the present invention had a high transparency to light in a near-infrared region of approximately 800 nm and greater, compared with a black compound of Synthesis Example 2, which is a bipyrrolinone compound of Example 6 of Japanese Unexamined Patent Application Publication No. 2009-84522 (PTL 1).

Note that the instrument and conditions used for the near-infrared transparency measurement are as follows.
Name of instrument: UV-Visible/NIR spectrophotometer V-770, manufactured by JASCO Corporation
Measurement Conditions Sample: powder (PSH-002 powder cell was used)
Measurement range: 300 to 2500 nm

In FIG. 1, the vertical axis represents a reflectance, which was used as an index of the transparency. Since the measurement of the transparency cannot be accomplished with the compound alone, the powder cell in a state in which no compound was loaded therein was assumed to have a reflectance of 100%, and an amount of light reflected by the cell in which the compound was loaded was calculated to determine the percentage representing the reflectance.

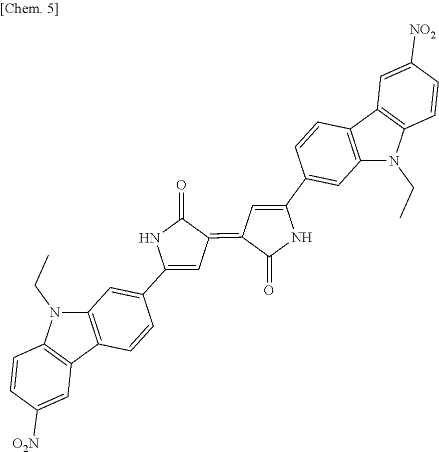 [SMILES: CCn1c2ccc([N+](=O)[O-])cc2c2ccc(C3=C/C(=C4/C=C(c5ccc6c7cc([N+](=O)[O-])ccc7n(CC)c6c5)NC4=O)C(=O)N3)cc21]

### Synthesis Example 2

20 g of carbazole and 6.0 g of succinic anhydride were mixed with 340 g of nitrobenzene under nitrogen purging, and then, 34 g of aluminum chloride was further added. The resulting mixture was stirred at room temperature for 4 hours and subsequently allowed to stand overnight. Thereafter, 68 ml of concentrated hydrochloric acid and 680 ml of water were added to the reaction mixture, which was then stirred at room temperature for 2 hours. The resulting precipitate, which was pale yellow, was filtered and washed with water, and subsequently, 20 g of potassium hydroxide and 800 g of water were added to the resulting wet cake, which was then stirred. Subsequently, an insoluble residue was filtered off, and the pH of the resulting filtrate was adjusted to approximately 2 with concentrated hydrochloric acid under stirring. The resulting precipitate, which was white, was filtered, washed with water, and dried to give 5.22 g of an intermediate 2. The reaction was performed twice. The obtained intermediate 2 had a purity of 87.7%.

Next, 6.49 g of the obtained intermediate 2 was mixed with a solution containing 3.74 g of ammonium acetate and 100 g of nitrobenzene, and the mixture was stirred at 90° C. for 2 hours and further stirred at 125° C. for 1 hour. The resulting mixture was heated at 200° C. for 4 hours to remove water therefrom, and subsequently, the resultant was cooled to give a blackish green precipitate. The resulting precipitate was washed and filtered repeatedly, with the washing being performed with 100 ml of acetone twice and further performed with 50 ml of methanol and water. The resulting wet cake was added to 30 ml of DMF, which was then refluxed for 2 hours. Subsequently, the mixture was washed with 50 ml of DMF, 100 ml of acetone, and 1500 ml of hot water and filtered, and the resulting solids were dried to give 2.6 g of a black compound. Electron impact mass spectrometry (MS-EI) was conducted, and the results confirmed that the compound had a molecular weight of 492 (m/z=492).

The obtained black compound was salt-milled with 10.85 g of sodium chloride and 2.5 g of diethylene glycol, the resultant was washed with water and filtered repeatedly, and the resulting wet cake was dried to give 2.2 g of a black compound of Comparative Example 2.

The infrared absorption of the obtained black compound was measured with the Fourier-transform infrared spectrophotometer (FT-IR), and the results were that an absorption peak corresponding to the NH linkage was observed at 3419 cm−1, and an absorption peak corresponding to the carbonyl linkage at 1668 cm−1.

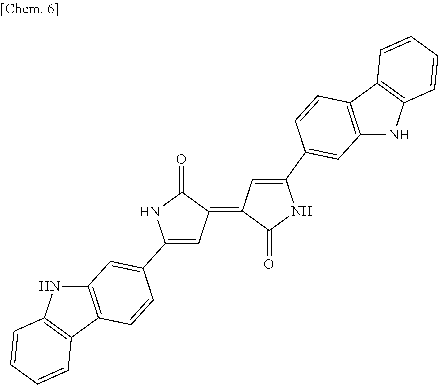 [SMILES: O=C1NC(c2ccc3c(c2)[nH]c2ccccc23)=C/C1=C1/C=C(c2ccc3c(c2)[nH]c2ccccc23)NC1=O]

### Synthesis Example 3

7.4 g of 3-nitromethyl carbazole and 3.0 g of succinic anhydride were mixed with 85 g of nitrobenzene under nitrogen purging, and 8.5 g of aluminum chloride was further added. The resulting mixture was stirred at room temperature for 2 hours, and subsequently, the contents were allowed to react at 50° C. for 4 hours. After the mixture was returned to room temperature, 17 ml of concentrated hydrochloric acid and 170 g of water were added to the reaction solution, which was then stirred at room temperature for 2 hours. Subsequently, the resultant was filtered, washed with water, and dried to give 5.32 g of an intermediate 3. The obtained intermediate 3 had a purity of 95.6%.

Next, 5.05 g of the obtained intermediate 3 was mixed with a solution containing 2.39 g of ammonium acetate and 50 g of nitrobenzene, and the mixture was stirred at 90° C. for 2 hours and further stirred at 125° C. for 1 hour. The resulting mixture was heated at 200° C. for 4 hours to remove water therefrom, and subsequently, the resultant was cooled to give a blackish green precipitate. The resulting precipitate was washed and filtered repeatedly, with the washing being performed with 100 ml of acetone twice and further performed with 50 ml of methanol and water. The resulting wet cake was added to DMF, which was then refluxed for 2 hours. Subsequently, the resultant was washed with DMF, acetone, and water and then dried to give 2.50 g of a black compound. The yield was 53%. Furthermore, electron impact mass spectrometry (MS-EI) was conducted, and the results confirmed that the compound had a molecular weight of 610 (m/z=610).

The obtained black compound was salt-milled with 12.50 g of sodium chloride and 2.91 g of diethylene glycol, the resultant was washed with water and filtered repeatedly, and the resulting wet cake was dried to give 2.30 g of a black compound of Example 2.

The infrared absorption of the obtained black compound was measured with the Fourier-transform infrared spectrophotometer (FT-IR), and the results were that an absorption peak corresponding to the NH linkage was observed at 3398 cm−1, and an absorption peak corresponding to the carbonyl linkage at 1673 cm−1, and an absorption peak corresponding to the nitro group at 1322 cm−1.

Next, a near-infrared transparency of the obtained black compound was measured with a spectrophotometer. The results are shown in FIG. 1, which indicates that the bipyrrolinone compound of the present invention had a high transparency to light in a near-infrared region of approximately 800 nm and greater.

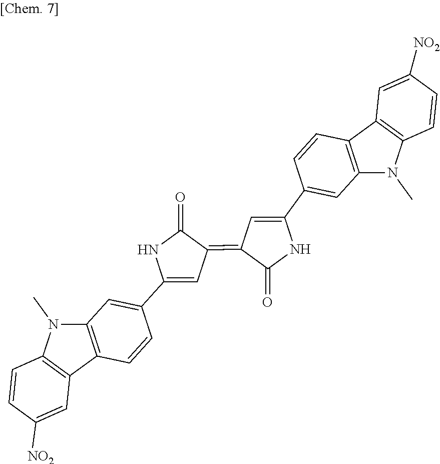 [SMILES: Cn1c2ccc([N+](=O)[O-])cc2c2ccc(C3=C/C(=C4/C=C(c5ccc6c7cc([N+](=O)[O-])ccc7n(C)c6c5)NC4=O)C(=O)N3)cc21]

### Example 1

### [Evaluation of Color Attributes]

1.6 g of the compound obtained in Synthesis Example 1, 6.4 g of a urethane-acrylic resin (solids concentration: 40%, solvent: xylene/isobutyl alcohol=50/50), 3.2 g of xylene, and 0.8 g of isobutyl alcohol were added to a glass vial, and further, glass beads having a diameter of 3 mm were added. The compound was dispersed with a paint conditioner for 1 hour. Subsequently, 20.0 g of the urethane-acrylic resin was added, and the compound was dispersed with the paint conditioner for 10 minutes. Thereafter, 7.5 g of the resulting dispersion, 10.6 g of the urethane-acrylic resin, 0.8 g of xylene, 0.2 g of isobutyl alcohol, and the glass beads were added to a glass vial, and the contents were dispersed for 10 minutes to form a coating composition of Example 1.

The obtained coating composition was applied to white art paper with an applicator to form a coating film having a film thickness of 25 μm. The obtained coating film was visually black. The color attributes of the coating film were measured with a spectrophotometer (name of instrument: DC650, from Datacolor), and the results were that L*=26.1, a*=0.03, and b*=0.10.

### [Evaluation of Reflectance]

The obtained coating composition was applied to white art paper with an applicator to form a coating film having a film thickness of 20 μm. The reflectance for light in the infrared region of the obtained coating film was measured with a spectrophotometer (name of instrument: V-770, from JASCO Corporation).

The evaluation criteria for a reflectance at 905 nm were as follows.
Reflectance of 80% or greater: A
Reflectance of 60 to 80%: B
Reflectance of 40 to 60%: C
Reflectance of 40% or less: D

The reflectance at 905 nm of the obtained coating film was rated as “A”, and an average reflectance over a range of 780 to 2500 nm of the coating film was 91.9%.

### Example 2

A coating composition of Example 2 was prepared as in Example 1, except that the black compound obtained in Synthesis Example 3 was used instead of the compound obtained in Synthesis Example 1.

The obtained coating composition was applied to white art paper with an applicator to form a coating film having a film thickness of 25 μm. The obtained coating film was visually gray. The color attributes of the coating film were measured with the spectrophotometer (name of instrument: DC650, from Datacolor). The results were that L*=26.0, a*=0.01, and b*=0.48.

### [Evaluation of Reflectance]

The reflectance for light in the infrared region of the obtained coating film having a film thickness of 20 μm was measured with the spectrophotometer as in Example 1. The reflectance at 905 nm of the obtained coating film was rated as “B”, and the average reflectance over a range of 780 to 2500 nm of the coating film was 89.6%.

### Comparative Example 1

A coating composition of Comparative Example 1 was prepared as in Example 1, except that carbon black (#2600 (trade name), manufactured by Mitsubishi Chemical Corporation) was used instead of the compound obtained in Synthesis Example 1.

The obtained coating composition was applied to white art paper with an applicator to form a coating film having a film thickness of 25 μm. The obtained coating film was visually gray. The color attributes of the coating film were measured with the spectrophotometer (name of instrument: DC650, from Datacolor), and the results were that L*=25.9, a*=−0.01, and b*=−0.79.

### [Evaluation of Reflectance]

The reflectance for light in the infrared region of the obtained coating film having a film thickness of 20 μm was measured with the spectrophotometer as in Example 1. The reflectance at 905 nm of the obtained coating film was rated as “D”, and the average reflectance over a range of 780 to 2500 nm of the coating film was 10.7%.

### Comparative Example 2

A coating composition of Comparative Example 2 was prepared as in Example 1, except that the black compound obtained in Synthesis Example 2 was used instead of the compound obtained in Synthesis Example 1.

The obtained coating composition was applied to white art paper with an applicator to form a coating film having a film thickness of 25 μm. The obtained coating film was visually gray. The color attributes of the coating film were measured with the spectrophotometer (name of instrument: DC650, from Datacolor). The results were that L*=25.7, a*=−0.03, and b*=−1.38.

### [Evaluation of Reflectance]

The reflectance for light in the infrared region of the obtained coating film having a film thickness of 20 μm was measured with the spectrophotometer as in Example 1. The reflectance at 905 nm of the obtained coating film was rated as “C”, and the average reflectance over a range of 780 to 2500 nm of the coating film was 82.9%.

### Comparative Example 3

A coating composition of Comparative Example 3 was prepared as in Example 1, except that a perylene black pigment (Paliogen Black S 0084 (trade name), manufactured by Sun Chemical Color & Effects) was used instead of the compound obtained in Synthesis Example 1.

The obtained coating composition was applied to white art paper with an applicator to form a coating film having a film thickness of 25 μm. The obtained coating film was visually gray. The color attributes of the coating film were measured with the spectrophotometer (name of instrument: DC650, from Datacolor), and the results were that L*=26.4, a*=−0.06, and b*=−1.06.

### [Evaluation of Reflectance]

The reflectance for light in the infrared region of the obtained coating film having a film thickness of 20 μm was measured with the spectrophotometer as in Example 1. The reflectance at 905 nm of the obtained coating film was rated as “A”, and an average reflectance over a range of 780 to 2500 nm of the coating film was 92.4%.

The above results demonstrate that the bipyrrolinone compound of the present invention has a low absorption for wavelengths in the infrared region and exhibits a high degree of blackness when used as a black colorant.

1. A compound represented by general formula (1) below, 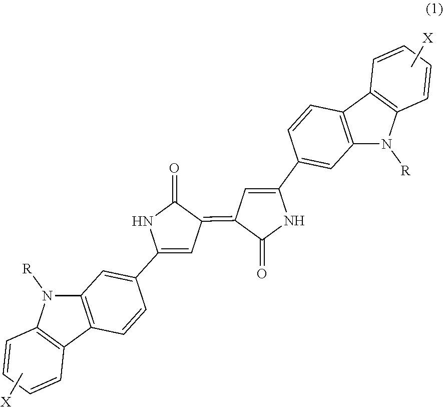
where R represents an alkyl group having 1 to 3 carbon atoms or represents an alkenyl group having 2 to 3 carbon atoms, and X represents one of a nitro group, a cyano group, a halogen, and an acetyl group.

2. The compound according to claim 1, wherein the compound is a bipyrrolinone compound.

3. The compound according to claim 1, wherein the compound is transparent to light having a wavelength of 800 to 1400 nm.

4. An ink, printed matter, a coating composition, a coated article, a plastic, a fiber, a film, a cosmetic, and a molded article that comprise the compound according to claim 1 and are transparent to light in a near-infrared region.

5. An ink, printed matter, a coating composition, a coated article, a plastic, a fiber, a film, a cosmetic, and a molded article that comprise the compound according to claim 3 and are transparent to light in a near-infrared region.

6. An ink, printed matter, a coating composition, a coated article, a plastic, a fiber, a film, a cosmetic, and a molded article that comprise the compound according to claim 1, the compound serving as a colorant, a coloring agent, or a pigment.

7. An ink, printed matter, a coating composition, a coated article, a plastic, a fiber, a film, a cosmetic, and a molded article that comprise the compound according to claim 3, the compound serving as a colorant, a coloring agent, or a pigment.

8. An ink, printed matter, a coating composition, a coated article, a plastic, a fiber, a film, a cosmetic, and a molded article that comprise the compound according to claim 1, the compound serving as a colorant, a coloring agent, or a pigment that is transparent to light in a near-infrared region.

9. An ink, printed matter, a coating composition, a coated article, a plastic, a fiber, a film, a cosmetic, and a molded article that comprise the compound according to claim 3, the compound serving as a colorant, a coloring agent, or a pigment that is transparent to light in a near-infrared region.
